(19) United States
(12) Patent Application Publication
(10) Pub. No.: US 2004/0034102 A1
(43) Pub. Date: Feb. 19, 2004

(54) Oral pediatric trimethobenzamide formulations and methods

(75) Inventors: Jefferson J. Gregory, Bristol, TN (US); Robert G. Bruns, Johnson City, TN (US); Dean R. Cirotta, Webster, NY (US); Thomas K. Rogers III, Bristol, TN (US); Charles L. Pamplin III, Chapel Hill, NC (US)

Correspondence Address: EDWARDS & ANGELL, LLP, P.O. BOX 9169, BOSTON, MA, 02209, US

(73) Assignee: King Pharmaceuticals, Inc., Bristol, TN (US)

(21) Appl. No.: 10/360,209

(22) Filed: Feb. 6, 2003

Related U.S. Application Data
(60) Provisional application No. 60/354,766, filed on Feb. 6, 2002.
(60) Provisional application No. 60/354,705, filed on Feb. 6, 2002.

(51) Int. Cl. A61K 31/165

(52) U.S. Cl. 514/622

(57) ABSTRACT

Oral pediatric trimethobenzamide compositions and methods for treating and controlling nausea and/or vomiting are disclosed in warm blooded animals, especially humans including children. The oral pediatric trimethobenzamide compositions and methods of the present invention are believed to be at least as effective as a 200 mg intramuscular (I.M.) trimethobenzamide HCl injectable formulation when administered at a dose of about 100 mg. In addition, an oral pediatric composition containing about 120 mg of trimethobenzamide HCl is believed to be uniquely approximately bioequivalent to a 200 mg intramuscular (I.M.) trimethobenzamide HCl injectable formulation when administered at a dose of about 100 mg.

### U.S. PATENT APPLICATION

This application for U.S. patent is filed as a provisional application under U.S.C., Title 35, §111(b).

### FIELD OF THE INVENTION

The present invention is concerned with oral pediatric trimethobenzamide compositions and methods useful for treating and controlling nausea and/or vomiting or emesis in warm-blooded animals, especially children.

### BACKGROUND

The process of nausea and vomiting is regulated by the chemoreceptor trigger zone (“CTZ”) which is located in the vomiting center. The vomiting center is located in the medulla. The chemoreceptor trigger zone is the primary trigger for emesis. Because the chemoreceptor trigger zone must first stimulate the vomiting center to induce emesis, the chemoreceptor trigger zone, by itself, cannot induce vomiting.

In addition, there are several receptors, e.g., 5-HT₃, D₂, H₁, Ach and opioid receptors, located in the chemoreceptor trigger zone, vomiting center and the GI tract which, when stimulated by neurotransmitters, such as serotonin (5-HT₃), dopamine (D₂), histamine (H₁), acetylcholine (Ach) and opioids (opioid), can induce the nausea and vomiting passageways.

Once the vomiting center is stimulated, the initial manifestations of the vomiting response often involves nausea, in which gastric tone is reduced, gastric peristalsis is reduced or absent and the tone of the duodenum and upper jejunum is increased, such that their contents reflux. Ultimately, the upper portion of the stomach relaxes while the pylorus constricts, and the coordinated contraction of the diaphragm and abdominal muscles leads to expulsion of gastric contents. Goodman and Gilman's The Pharmacological Basis of Therapeutics, 9th Edition, McGraw-Hill Health Care Divisions, New York, pp. 928 (1995).

Nausea and vomiting are common symptoms in postoperative patients and in people undergoing chemotherapy and radiation treatment. Nausea and vomiting are also common during pregnancy and in people inflicted with gastroenteritis, uremia, electrolyte and endocrine disturbances or in people who have been administered other chemical emetic agents. In addition to these blood-borne emetic substances, nausea and vomiting can be induced in people by afferent stimulation, such as motion, pain, psychological conditions, tactile pharyngeal impulses, distention, intracranial pressure and labyrinthine disturbances.

Trimethobenzamide hydrochloride is a prescription drug that has been available in the market since about the 1960s. It is used to treat and control nausea and vomiting. Trimethobenzamide hydrochloride is N-[2-(dimethylamino)-ethoxy]-3,4,5trimethoxybenzamide hydrochloride, and it has a molecular weight of about 424.93. The chemical structure of trimethobenzamide hydrochloride is (CH3)₂ N—CH₂ CH₂ O CH₂ NHC OCH₃ OCH₃ OCH₃ O.HCl or:

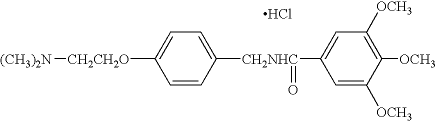

According to the FDA, trimethobenzamide hydrochloride is effective for postoperative nausea and vomiting and nausea associated with gastroenteritis, and it is generally prescribed for patients with the “flu” and other illnesses or conditions. Trimethobenzamide is believed to control or alleviate nausea and vomiting by (1) inhibiting emetic stimulation of the chemoreceptor trigger zone in the medulla oblongata through which emetic impulses are conveyed to the vomiting center and (2) antagonizing D₂ dopamine and 5-HT₃ serotonin receptors. Even though trimethobenzamide hydrochloride has been widely available for many years, the only routes and dosage forms that have been approved by the FDA are: 100 mg and 250 mg capsules; 100 mg and 200 mg suppositories; and 100 mg/ml in 2-ml ampules and prefilled syringes and in 20-ml vials as injectables. The injectable form is intended for intramuscular administration only; it is not recommended for intravenous use.

The usual oral dosage of trimethobenzamide hydrochloride is one capsule (100-milligrams or 250-milligrams) taken 3 or 4 times per day, as determined by the doctor. The recommended rectal dosage is 1 suppository (100-milligrams or 200-milligrams) inserted into the rectum 3 or 4 times per day, as determined by the doctor. The recommended injectable dosage is 2-milliliters (200-milligrams) 3 or 4 times per day, intramuscularly. Following administration, it is believed that approximately 30%-50% of the drug is excreted unchanged in the urine in 48-72 hr. The time/action profile of trimethobenzamide hydrochloride for controlling nausea and vomiting that has been reported is:

| | ONSET | PEAK | DURATION |
|---|---|---|---|
| PO | 10-40 min | unknown | 3-4 hr |
| IM | 15-35 min | unknown | 2-3 hr |
| Rect | 10-40 min | unknown | 3-4 hr |

Most prescription drugs placed on the market are given trade names (also called proprietary, brand, or specialty names) to distinguish them as being produced and marketed exclusively by a particular manufacturer. In the United States, these names are usually registered as trademarks with the Patent Office; this gives the registrant certain legal rights with respect to the names' use. A trade name may be registered for a product containing a single active ingredient, with or without additives.

Trimethobenzamide hydrochloride is no different. Since its introduction into the market place many decades ago as a prescription drug, trimethobenzamide hydrochloride has been marketed under various brand names, e.g., Arrestin, Benzacot, Brogan, Stemetic, Tebamide, Tegamide, T-Gen, Ticon, Tigan®, Tiject-20, Triban, Tribenzagan, and Trimazide.

One of the most recognized brand names for trimethobenzamide hydrochloride is Tigan®. It has been available in capsule, suppository and injection dosage forms. Each Tigan® capsule for oral use, with opaque blue cap and opaque white body, contains trimethobenzamide hydrochloride equivalent to either 100 mg or 250 mg. Each Tigan® capsule also includes FD&C Blue No. 1, FD&C Red No. 3, lactose, magnesium stearate, starch and titanium dioxide, as the inactive ingredients. Each Tigan® suppository contains either 100 mg or 200 mg trimethobenzamide hydrochloride and 2% benzocaine in a base compounded with polysorbate 80, white beeswax and propylene glycol monostearate. Each 2 mL Tigan® ampul for intramuscular injection contains 200 mg trimethobenzamide hydrochloride compounded with 0.2% parabens (methyl and propyl) as preservatives, 1 mg sodium citrate and 0.4 mg citric acid as buffers and pH adjusted to approximately 5.0 with sodium hydroxide. Each Tigan® multi-dose vial for intramuscular injection contains 100 mg trimethobenzamide hydrochloride per milliliter compounded with 0.45% phenol as preservative, 0.5 mg sodium citrate and 0.2 mg citric acid as buffers and pH adjusted to approximately 5.0 with sodium hydroxide. Each Tigan® disposable syringe contains 200 mg trimethobenzamide hydrochloride per 2 milliliters compounded with 0.45% phenol as preservative, 1 mg sodium citrate and 0.4 mg citric acid as buffers, 0.2 mg disodium edetate as stabilizer and pH adjusted to approximately 5.0 with sodium hydroxide.

In 1979, however, the FDA published notice in the Federal Register, dated Jan. 9, 1979, to advise the public that trimethobenzamide capsules containing 100 mg and 250 mg are not approximately bioequivalent to a 200-milligram intramuscular dose and do not achieve plasma levels necessary to effectively treat or control nausea and vomiting. The FDA January 9^{th} notice further advised the public that trimethobenzamide capsules containing 100 mg and 250 mg must be reformulated to 200 mg and 400 mg, respectively, to achieve approximate bioequivalence to a 200-milligram intramuscular dose. More specifically, the FDA notice in the Jan. 9, 1979 Federal Register stated that:

This notice . . . states that to obtain effective plasma levels for these drug products, a dosage of 200 milligrams intramuscularly or 400 milligrams orally is required, and that as part of the marketing conditions for the capsule dosage form, the capsules now containing 100 milligrams or 250 milligrams must be reformulated to 200 milligrams or 400 milligrams, respectively, . . .

ORAL AND PARENTERAL TRIMETHOBENZAMIDE ARE NOT BIOEQUIVALENT. AN ORAL DOSE OF 400 MILLIGRAMS OF TRIMETHOBENZAMIDE YIELDS PLASMA LEVELS APPROXIMATELY EQUIVALENT TO A 200-MILLIGRAM INTRAMUSCULAR DOSE. The systemic bioavailability of orally administered trimethobenzamide is about 60 percent of the bioavailability of intramuscularly administered drug, possibily because of slow absorption and rapid liver metabolism (first pass effect). This difference is manifested as diminished peak blood levels and a diminished area under the plasma concentration curve following oral, as compared to parenteral administration.

Notwithstanding this FDA notice, which was published more than 23 years ago, there is no oral trimethobenzamide dose available today which is approximately bioequivalent to a 200-milligram intramuscular dose or which achieves plasma levels effective to treat or control nausea and vomiting. In addition, there is no oral pediatric trimethobenzamide dose available today which is approximately bioequivalent to a 100-milligram intramuscular dose or which achieves plasma levels effective to treat or control nausea and vomiting in pediatric patients. Given the fact that the FDA has determined that the capsules containing 100 mg and 250 mg of trimethobenzamide must be reformulated, there is a definite need for an oral trimethobenzamide dose which is approximately bioequivalent or superior to a 200-milligram intramuscular dose, i.e., which can achieve an effective plasma level, for treating and controlling nausea and/or vomiting, especially postoperative nausea and vomiting and nausea associated with gastroenteritis, the indications approved by the FDA. There is also a definite need for an oral pediatric trimethobenzamide dose which is approximately bioequivalent or superior to a 100-milligram intramuscular dose, i.e., which can achieve an effective plasma level, for treating and controlling nausea and/or vomiting in pediatric patients.

### SUMMARY OF THE INVENTION

The present invention overcomes and alleviates the above-mentioned drawbacks and disadvantages in the trimethobenzamide art through the discovery of novel oral trimethobenzamide compositions and methods useful for treating and controlling nausea and/or vomiting in warm-blooded animals, especially humans including pediatrics.

Generally speaking, the oral trimethobenzamide compositions and methods of the present invention are at least as effective as an FDA-approved 200 mg intramuscular (I.M.) trimethobenzamide HCl injectable formulation. In other words, the oral trimethobenzamide compositions and methods of the present invention achieve effective plasma (exposure) levels for treating and controlling nausea and/or vomiting, which are at least approximately equal to or greater than those plasma (exposure) levels achieved by a FDA-approved 200 mg intramuscular (I.M.) trimethobenzamide HCl injectable formulation when administered at a 200 mg dose. Moreover, following oral administration, the oral trimethobenzamide compositions and methods of the present invention reach maximum concentration and elimination of trimethobenzamide at a rate that is very similar for the intramuscular (I.M.) dosage form.

Quite amazingly, it has been discovered that an oral dose of about 300 mg of trimethobenzamide is uniquely approximately bioequivalent to a 200 mg intramuscular (I.M.) trimethobenzamide HCl injectable formulation, whereas an oral dose of about 400 milligrams of trimethobenzamide is not. The FDA 1979 public notice in the Federal Register, notwithstanding, it has been discovered, quite unexpectedly, that the bioequivalency (PK) parameters of an oral dose of about 400 mg of trimethobenzamide are uniquely approximately at least about 20% greater than the corresponding bioequivalency (PK) parameters for a 200 mg intramuscular (I.M.) trimethobenzamide HCl injectable formulation.

More specifically, it has been surprisingly discovered that the mean maximum concentration, Cₘₐₓ, was comparable following the administration of the 200 mg I.M. injection and a capsule containing about 300 mg, with mean±SD maximum plasma trimethobenzamide concentrations of 3728.79±997.385 mcg/L and 3816.94±1355.016 mcg/L for the 200 mg I.M. injection and 300 mg capsule, respectively. Following the administration of the 4×100 mg capsules and a capsule containing about 400 mg, however, the mean Cₘₐₓ was approximately 39% greater than that following the 200 mg I.M. injection, with mean±SD maximum plasma trimethobenzamide concentrations of 5197.73±1534.570 mcg/L and 5211.23±1788.106 mcg/L, for the 4×100 mg capsules and the 400 mg capsule, respectively.

It has also been surprisingly discovered that the measures of exposure, AUCₗₐₛₜ and AUC_{0-inf}, were comparable for the 200 mg I.M. injection and a capsule containing about 300 mg, as evidenced by mean±SD values for AUCₗₐₛₜ of 10123.78±1708.292 mcg*hr/L and 9460.65±2429.683 mcg*hr/L and AUC_{0-inf} of 10465.00±1807.,731 mcg*hr/L and 10218.11±2690.333 mcg*hr/L for the 200 mg I.M. injection and the 300 mg capsule, respectively. The mean AUCₗₐₛₜ and AUC_{0-inf} following the administration of the 4×100 mg capsules and a capsule containing about 400 mg were approximately 20% greater than that following the 200 mg I.M. injection, with mean±SD values for AUCₗₐₛₜ of 12426.04±3335.331 mcg*hr/L and 12667.77±3433.118 mcg*hr/L and AUC_{0-inf} of 13493.38±3694.251 mcg*hr/L and 13647.39±3760.144 mcg*hr/L for the 4×100 mg capsules and the 400 mg capsule, respectively.

Still further, it was surprisingly discovered that the elimination half-life, T_{1/2}, was similar across all dosage forms with mean±SD values for the 200 mg I.M., 300 mg capsule, 400 mg capsule, and 4×100 mg capsules of 6.8±1.74 hr, 7.8±2.37 hr, 7.4±2.02 hr, and 8.0±2.32 hr, respectively.

Thus, the unique oral trimethobenzamide formulations and methods of the present invention, when orally administered to warm-blooded animals, especially humans, generate results considerably superior to those obtained with oral trimethobenzamide formulations available heretofore, at least in terms of enhanced drug bioavailability, plasma (exposure) levels and effectiveness in treating and controlling nausea and/or vomiting.

While the present invention contemplates any oral trimethobenzamide dosage form, such as capsules, caplets, tablets, powders and liquids, and strength which is at least as effective as an FDA-approved 200 mg intramuscular (I.M.) trimethobenzamide HCl injectable, preferred oral trimethobenzamide formulations are in capsule form and have dosage strengths in the range of from about 300 mg to about 400 mg or more, such as 325 mg, 350 mg, 375 mg, 400 mg, 425 mg, 450 mg etc.

An especially preferred oral trimethobenzamide dosage form in accordance with the present invention is a capsule which contains about 300 mg of trimethobenzamide and which is approximately bioequivalent to a 200 mg intramuscular (I.M.) trimethobenzamide HCl injectable formulation when administered orally to a warm-blooded animal, especially a human.

To illustrate that the oral trimethobenzamide formulations of the present invention are at least approximately bioequivalent or superior in effectiveness to a FDA-approved 200 mg intramuscular (I.M.) trimethobenzamide HCl injectable formulation and superior in effectiveness to the oral trimethobenzamide formulations available on the market heretofore, Mean PK parameters evaluated and estimated PK parameters are reported in Table 1 below. Specifically, Mean PK parameters evaluated for a FDA-approved 200 mg intramuscular (I.M.) trimethobenzamide HCl injectable and for oral trimethobenzamide formulations of the present invention having dosage strengths of about 300 mg and about 400 mg, and estimated Mean PK parameters for oral trimethobenzamide formulations of the present invention having dosage strengths of about 325 mg, about 350 mg, about 375 mg doses, are compared in Table 1 below.

TABLE 1
Mean PK Parameters

| | | | AUC 0-inf | |
|---|---|---|---|---|
| | Cmax (mcg/L) | Tmax (hr) | (mcg*hr/L) | FAUC0-inf |
| 200 mg I.M. | 3728.79 | 0.54 | 10465.00 | N/A |
| evaluated | | | | |
| 300 mg CAP | 3816.94 | 0.78 | 10218.11 | 0.65 |
| evaluated | | | | |
| 325 mg CAP | 4165.51 | 0.76 | 11075.41 | 0.65 |
| estimated | | | | |
| 350 mg CAP | 4514.09 | 0.76 | 11932.71 | 0.65 |
| estimated | | | | |
| 375 mg CAP | 4862.66 | 0.76 | 12790.00 | 0.65 |
| estimated | | | | |
| 400 mg CAP | 5211.23 | 0.73 | 13647.30 | 0.65 |
| evaluated | | | | |
| Pediatric | | | | |
| 100 mg I.M. | 1864.40 | 0.54 | 5232.50 | N/A |
| evaluated | | | | |
| 120 mg CAP | 1908.47 | 0.78 | 5109.06 | 0.65 |
| estimated | | | | |
| 125 mg CAP | 1961.48 | 0.78 | 5250.97 | 0.65 |
| estimated | | | | |
| 130 mg CAP | 2014.50 | 0.78 | 5392.89 | 0.65 |
| estimated | | | | |
| 140 mg CAP | 2120.52 | 0.78 | 5676.73 | 0.65 |
| estimated | | | | |
| 150 mg CAP | 2226.55 | 0.78 | 5960.56 | 0.65 |
| estimated | | | | |
| 160 mg CAP | 2332.57 | 0.78 | 6244.40 | 0.65 |
| estimated | | | | |
| 175 mg CAP | 2491.61 | 0.78 | 6670.16 | 0.65 |
| estimated | | | | |
| 180 mg CAP | 2544.63 | 0.78 | 6812.07 | 0.65 |
| estimated | | | | |
| 200 mg CAP | 2756.68 | 0.78 | 7379.75 | 0.65 |
| estimated | | | | |

Further, it is also believed that oral pediatric trimethobenzamide compositions and methods of the present invention are at least as effective as a FDA-approved intramuscular (I.M.) trimethobenzamide HCl injectable formulation when administered at about a 100 mg dose. In other words, it is believed that oral pediatric trimethobenzamide compositions and methods of the present invention will achieve effective plasma (exposure) levels for treating and controlling nausea and/or vomiting in pediatric patients, which are at least approximately equal to those plasma (exposure) levels achieved by a FDA-approved intramuscular (I.M.) trimethobenzamide HCl injectable formulation when administered at a dose of about 100 mg. Moreover, it believed that an oral pediatric dose of about 120 mg of trimethobenzamide in accordance with the present invention is uniquely approximately bioequivalent to a FDA-approved 200 mg intramuscular (I.M.) trimethobenzamide HCl injectable formulation when administered at a dose of about 100 mg.

As evidence of pediatric bioequivalency, the estimated mean PK parameters for an oral pediatric dose of about 120 mg of trimethobenzamide in accordance with the present invention are comparable to the mean PK parameters for a FDA-approved 200 mg intramuscular (I.M.) trimethobenzamide HCl injectable formulation when administered at a dose of about 100 mg. See Table 1 above. More specifically, the estimated mean maximum concentration, Cₘₐₓ, for an oral pediatric dose of about 120 mg of trimethobenzamide in accordance with the present invention is about 1908.47 mcg/L, whereas the mean maximum concentration, Cₘₐₓ for a FDA-approved 200 mg intramuscular (I.M.) trimethobenzamide HCl injectable formulation when administered at a dose of about 100 mg is about 1864.40 mcg/L. In addition, the mean Tmax(hr) and mean AUC 0-inf (mcg*hr/L) are comparable for a FDA-approved 200 mg intramuscular (I.M.) trimethobenzamide HCl injectable formulation when administered at a dose of about 100 mg and a capsule containing about 120 mg. According to Table 1 above, the mean Tmax(hr) and AUCₗₐₛₜ values for a FDA-approved 200 mg intramuscular (I.M.) trimethobenzamide HCl injectable formulation when administered at a dose of about 100 mg are about 0.54 hrs and about 5232.50 mcg*hr/L, respectively, and the estimated mean Tmax(hr) and mean AUC 0-inf (mcg*hr/L) for an oral pediatric dose of about 120 mg of trimethobenzamide in accordance with the present invention are about 0.78 hrs and about 5109.06 mcg*hr/L, respectively.

Thus, it is believed that the unique oral pediatric trimethobenzamide formulations and methods of the present invention, when orally administered to children will generate results considerably superior to or greater than those obtained with oral pediatric trimethobenzamide formulations available heretofore, at least in terms of enhanced drug bioavailability, plasma (exposure) levels and effectiveness in treating and controlling nausea and/or vomiting in pediatric patients. Moreover, while the present invention contemplates any oral pediatric trimethobenzamide dosage form, such as capsules, caplets, tablets, powders and liquids, and any strength which is at least as effective as a FDA-approved intramuscular (I.M.) trimethobenzamide HCl injectable formulation when administered at a dose of about 100 mg, such as 120 mg, 125 mg, 130 mg, 140 mg, 150 mg, 160 mg, 175 mg, 180 mg, 200 mg, 220 mg and 240 mg oral pediatric strengths, an especially preferred oral pediatric trimethobenzamide dosage formulation is one which is in capsule form and has a dosage strength of about 120 mg of trimethobenzamide and which is approximately bioequivalent to a FDA-approved intramuscular (I.M.) trimethobenzamide HCl injectable formulation when administered at a dose of about 100 mg.

Also in accordance with the present invention, the oral trimethobenzamide formulations are stable for at least about 24 months or more and are virtually, if not completely, free of impurities, such as trimethobenzoic acid and trimethobenzamide oxidation products.

The present invention also contemplates methods of treating and controlling nausea and/or vomiting in warm-blooded animals, especially humans including pediatrics. In accordance with the present invention, the oral trimethobenzamide dosage forms are orally administered, preferably in capsule form, as a single capsule or other single oral dosage forms three or four times daily as needed, according to the prescribing physician, to treat and control nausea and/or vomiting. Such methods also contemplate orally administering trimethobenzamide dosage forms of the present invention at dosage strengths, preferably in capsule form, as a single capsule or other single oral dosage forms, three or four times daily as needed, so as to achieve plasma (exposure) levels that are approximately comparable or superior to a 200 mg intramuscular (I.M.) trimethobenzamide HCl injectable formulation when administered at a dose of about 200 mg in an adult or when administered at a dose of about 100 mg in a child, respectively, to treat and control nausea and/or vomiting in adults and children.

Also in accordance with the present invention, the oral pediatric trimethobenzamide dosage forms are orally administered, preferably in capsule form, as a single capsule or other single oral dosage forms, as follows: children who weigh 30 to 90 lbs: one or two pediatric strength capsules, e.g., 120 mg, etc., t.i.d. or q.i.d, as needed, according to the prescribing pediatrician.

The present invention also contemplates methods of instructing patients to treat and control nausea vomiting in warm-blooded animals, especially humans including pediatric patients. In accordance with the present invention, patients are instructed to take oral trimethobenzamide dosage forms of the present invention, preferably in capsule form, as a single capsule or other single oral dosage forms, three or four times daily as needed to treat and control nausea and/or vomiting. Also in accordance with the present invention, patients are instructed to take oral trimethobenzamide dosage forms of the present invention at a dosage strength, preferably in capsule form, as a single capsule or other single oral dosage forms, three or four times daily as needed, so as to achieve plasma (exposure) levels that are approximately comparable or superior to a 200 mg intramuscular (I.M.) trimethobenzamide HCl injectable formulation when administered at a dose of about 200 mg in an adult or when administered at a dose of about 100 mg in a child, respectively, to treat and control nausea and/or vomiting in adults and children.

Thus, it should now be apparent to those versed in this art that the present invention contemplates any oral trimethobenzamide formulation in any dosage form and in any strength which is at least approximately bioequivalent or superior to a 200 mg intramuscular (I.M.) trimethobenzamide HCl injectable formulation in treating and controlling nausea and/or vomiting in warm-blooded animals, especially a humans. It should also now be apparent to those versed in this art that the present invention contemplates any oral pediatric trimethobenzamide formulation in any dosage form and in any strength which is at least approximately bioequivalent or superior to a FDA-approved 200 mg intramuscular (I.M.) trimethobenzamide HCl injectable formulation when administered at a dose of about 100 mg to treat and control nausea and/or vomiting in children.

In addition, the oral trimethobenzamide compositions of the present invention may be formulated by compounding trimethobenzamide with any suitable pharmaceutical excipients, such as lactose, magnesium stearate, starch, dibasic calcium phosphate and microcrystalline cellulose, to form a blend, which may then be used to produce an oral dosage form, such as a capsule, tablet, caplet, powder or liquid.

Accordingly, it is an object of the present invention to provide novel oral dosage forms and methods that overcome the shortcomings of the prior art and fully satisfies the critical and unfilled need for oral trimethobenzamide dosage forms.

Another object of the present invention is to provide oral dosage forms and methods that can deliver an effective antiemetic-antinausea trimethobenzamide amount thereby substantially fulfilling the pressing need of the prior art.

Another object of the present invention is to provide oral dosage forms and methods that can deliver antiemetic-antinausea trimethobenzamide amounts that will achieve plasma (exposure) levels which are at least approximately equal to or greater than those achieved by a FDA-approved 200 mg intramuscular (I.M.) trimethobenzamide HCl injectable formulation. Also, an object of the present invention is to provide oral pediatric dosage forms and methods that can deliver antiemetic-antinausea pediatric trimethobenzamide amounts that will achieve plasma (exposure) levels which are at least approximately equal to those achieved by a FDA-approved 200 mg intramuscular (I.M.) trimethobenzamide HCl injectable formulation when at a dose of about 100 mg.

Another object of the instant invention is to provide oral dosage forms and methods that can deliver antiemetic-antinausea trimethobenzamide amounts that are at least as effective as a 200 mg intramuscular (I.M.) trimethobenzamide HCl injectable formulation in treating and controlling nausea and/or vomiting in warm-blooded animals, especially humans.

Another object of the present invention is to provide oral dosage forms and methods that can deliver antiemetic-antinausea trimethobenzamide amounts that are uniquely approximately bioequivalent to a 200 mg intramuscular (I.M.) trimethobenzamide HCl injectable formulation.

Another object of the instant invention is to provide oral pediatric dosage forms and methods that can deliver antiemetic-antinausea trimethobenzamide amounts that are at least as effective a FDA-approved 200 mg intramuscular (I.M.) trimethobenzamide HCl injectable formulation when administered at a dose of about 100 mg to treat and control nausea and/or vomiting in children.

Another object of the present invention is to provide oral pediatric dosage forms and methods that can deliver antiemetic-antinausea trimethobenzamide amounts that are uniquely approximately bioequivalent to a FDA-approved 200 mg intramuscular (I.M.) trimethobenzamide HCl injectable formulation when administered at a dose of about 100 mg.

Another object of the present invention is to provide oral dosage forms and methods that can deliver antiemetic-antinausea trimethobenzamide amounts useful for effectively treating and controlling emesis and nausea induced in warm-blooded animals, especially humans including pediatric patients, by blood-borne emetic substances or by afferent stimulation.

Another object of the instant invention is to provide oral dosage forms and methods that can deliver antiemetic-antinausea trimethobenzamide amounts useful for effectively treating and controlling emesis and nausea in postoperative patients and for effectively treating and controlling gastroenteritis-induced nausea.

Another object of the instant invention is to provide oral dosage forms and methods that can deliver antiemetic-antinausea trimethobenzamide amounts useful for effectively treating and controlling chemotherapy-induced emesis and nausea.

Another object of the present invention is to provide oral dosage forms and methods that can deliver antiemetic-antinausea trimethobenzamide amounts useful for effectively treating and controlling radiation-induced emesis and nausea.

Another object of the present invention is to provide oral dosage forms and methods that can deliver antiemetic-antinausea trimethobenzamide amounts that are immediately released to provide instant antiemetic-antinausea therapy to a warm-blooded animal, especially humans including children, in need of same.

Another object of the instant invention is to provide oral antiemetic-antinausea trimethobenzamide dosage forms that can be provided in capsule form for oral consumption by warm-blooded animals, especially humans including children, for treating and controlling nausea and/or vomiting.

Another object of the present invention is to provide oral dosage forms and methods that can deliver effective antiemetic-antinausea amounts of trimethobenzamide that are safe and well tolerated.

These and other objects, features, and advantages of the present invention may be better understood and appreciated from the following detailed description of the embodiments thereof, selected for purposes of illustration and shown in the accompanying drawing, detailed description and examples. It should therefore be understood that the particular embodiments illustrating the present invention are exemplary only and not to be regarded as limitations of the present invention.

### BRIEF DESCRIPTION OF THE FIG.

The foregoing and other objects, advantages and features of the invention, and the manner in which the same are accomplished, will become more readily apparent upon consideration of the following detailed description of the invention taken in conjunction with the accompanying FIG., which illustrates a preferred and exemplary embodiment, wherein:

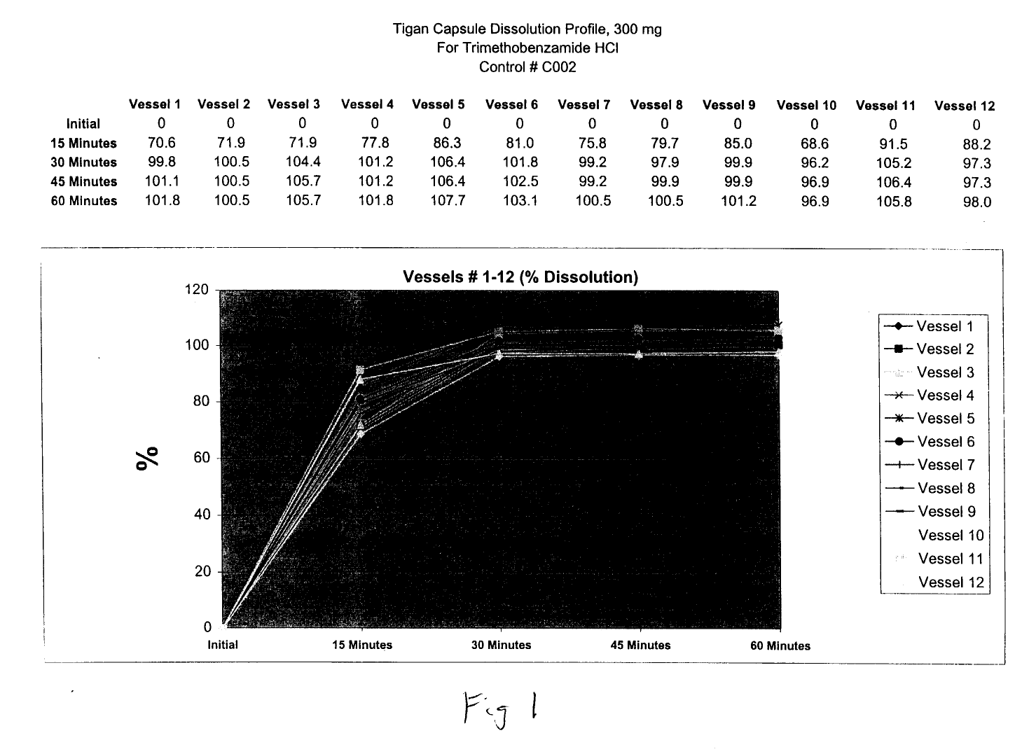

FIG. 1 is a dissolution profile of an oral capsule containing about 300 mg of trimethobenzamide hydrochloride compounded with lactose, magnesium stearate and starch, in accordance with the present invention.

### DETAILED DESCRIPTION OF THE INVENTION

By way of illustrating and providing a more complete appreciation of the present invention and many of the attendant advantages thereof, the following detailed description is given concerning the novel oral trimethobenzamide compositions and methods useful for treating and controlling nausea and vomiting in warm-blooded animals, especially humans including children.

Any pharmaceutically acceptable form of trimethobenzamide can be employed, i.e., the free base or a pharmaceutically acceptable salt thereof, e.g., trimethobenzamide hydrochloride, trimethobenzamide hydrobromide, trimethobenzamide acetate, trimethobenzamide hydrate, trimethobenzamide mesylate, etc.

The trimethobenzamide can be conveniently administered orally to warm-blooded animals to elicit a systemic, therapeutically anti-nausea or anti-emetic response by formulating it into an oral dosage form comprising trimethobenzamide, in a systemic, therapeutically effective anti-nausea or anti-emetic amount, together with a nontoxic pharmaceutically acceptable oral carrier thereof As indicated earlier, trimethobenzamide can be employed in the form of the free base or in the form of a pharmaceutically acceptable salt. Suitable nontoxic pharmaceutically acceptable oral excipients or carriers, such as lactose, magnesium stearate, starch, dibasic calcium phosphate, and microcrystalline cellulose, will be apparent to those skilled in the art of oral pharmaceutical formulations. For those not skilled in the art, reference is made to the text entitled “Remington's Pharmaceutical Sciences”, 20^{th} edition, 2000, which is incorporated herein by reference in its entirety. It should be understood that the choice of suitable carriers will depend on the exact nature of the particular oral dosage form selected or required, e.g., whether the trimethobenzamide is to be formulated into an oral capsule, oral tablet, oral caplet, oral liquid or other oral forms. A preferred oral dosage form in accordance with the present invention is a capsule which will contain trimethobenzamide, especially trimethobenzamide hydrochloride, compounded with lactose, magnesium stearate and starch as the pharmaceutical excipients. It should therefore be understood that the present invention envisions the use of any form of trimethobenzamide and any suitable pharmaceutical excipients to produce oral trimethobenzamide dosage forms to accomplish the objectives of the present invention.

Those skilled in the art will be aware that a systemic, therapeutically effective anti-nausea or anti-emetic amount of trimethobenzamide may vary with the age, size, weight and general physical condition of the patient. Typically the dosage level will be more similar to the expected dosage level for intravenous administration than to the dosage levels currently employed for other methods of administration, for example oral, rectal or subcutaneous.

As a practical matter the selected therapeutic compositions will normally be prepared in dosage unit forms to contain systemic, therapeutically effective amounts of trimethobenzamide. In specific instances, fractions of the dosage units or multiple dosage units may be employed. Typically, dosage units may be prepared to deliver at least about 300 mg to about 400 mg or more of trimethobenzamide per capsule or other oral unit dosage forms (e.g., 300 mg, 325 mg, 350 mg, 375 mg, 400 mg, 425 mg, 450 mg, etc.), these being the preferred types of compositions. With respect to children, dosage units may be prepared to deliver at least about 120 mg to about 200 mg or more of trimethobenzamide per capsule or other oral unit dosage forms (e.g., 125 mg, 130 mg, 140 mg, 150 mg, 160 mg, 175 mg, 180 mg, 200 mg, 220 mg, 240 mg, etc.), these being the preferred types of pediatric compositions.

Exemplary oral trimethobenzamide capsule formulations of the present invention are illustrated in Table 2 below. The amounts recited for each of the oral trimethobenzamide formulations set forth in Table 2 are approximate amounts and are based upon the Standard Production Formula reported in Table 7 in Example V below.

TABLE 2
Oral Trimethobenzamide Formulations

| | Trimethobenzamide | | Magnesium | | Weight per |
|---|---|---|---|---|---|
| Trimethobenzamide | HCl | Lactose | Stearate | Starch | Capsule |
| Capsule Strength | | | | | |
| 300 mg | 0.3029 g | 0.0432 g | 0.0060 g | 0.0918 g | 0.4440 g |
| 325 mg | 0.3282 g | 0.0468 g | 0.0065 g | 0.0995 g | 0.4810 g |
| 350 mg | 0.3535 g | 0.0504 g | 0.0070 g | 0.1071 g | 0.5180 g |
| 375 mg | 0.3878 g | 0.0540 g | 0.0075 g | 0.1148 g | 0.5550 g |
| 400 mg | 0.4039 g | 0.0576 g | 0.0080 g | 0.1224 g | 0.5920 g |
| 425 mg | 0.4292 g | 0.0612 g | 0.0085 g | 0.1301 g | 0.6290 g |
| 450 mg | 0.4545 g | 0.0648 g | 0.0090 g | 0.1377 g | 0.6660 g |
| Pediatric Strength | | | | | |
| 120 mg | 0.1212 g | 0.0173 g | 0.0024 g | 0.0367 g | 0.1776 g |
| 125 mg | 0.1262 g | 0.0180 g | 0.0025 g | 0.0383 g | 0.1850 g |
| 130 mg | 0.1313 g | 0.0187 g | 0.0026 g | 0.0398 g | 0.1924 g |
| 140 mg | 0.1414 g | 0.0202 g | 0.0028 g | 0.0428 g | 0.2072 g |
| 150 mg | 0.1515 g | 0.0216 g | 0.0030 g | 0.0459 g | 0.2220 g |
| 160 mg | 0.1616 g | 0.0230 g | 0.0032 g | 0.0490 g | 0.2368 g |
| 175 mg | 0.1767 g | 0.0252 g | 0.0035 g | 0.0536 g | 0.2590 g |
| 180 mg | 0.1818 g | 0.0259 g | 0.0036 g | 0.0552 g | 0.2664 g |
| 200 mg | 0.2020 g | 0.0288 g | 0.0040 g | 0.0612 g | 0.2960 g |
| 220 mg | 0.2222 g | 0.0317 g | 0.0044 g | 0.0673 g | 0.3256 g |
| 240 mg | 0.2424 g | 0.0346 g | 0.0048 g | 0.0735 g | 0.3552 g |

As indicated above, the oral trimethobenzamide compositions and methods of the present invention are at least as effective as a FDA-approved 200 mg intramuscular (I.M.) trimethobenzamide HCl injectable formulation. The oral trimethobenzamide compositions and methods of the present invention not only achieve plasma (exposure) levels which are at least approximately equal to those achieved by a FDA-approved 200 mg intramuscular (I.M.) trimethobenzamide HCl injectable formulation, but the time to reach maximum concentration and the elimination of trimethobenzamide are similar for the oral and intramuscular (I.M.) dosage forms. In addition, an oral dose of about 300 mg of trimethobenzamide of an oral formulation in accordance with the present invention is uniquely approximately bioequivalent to a 200 mg intramuscular (I.M.) trimethobenzamide HCl injectable formulation.

Also as indicated above, the oral pediatric trimethobenzamide compositions and methods of the present invention are at least as effective as a FDA-approved 200 mg intramuscular (I.M.) trimethobenzamide HCl injectable formulation when administered at about a 100 mg dose. The oral pediatric trimethobenzamide compositions and methods of the present invention not only achieve plasma (exposure) levels which are at least approximately equal to or greater than those achieved by a FDA-approved 200 mg intramuscular (I.M.) trimethobenzamide HCl injectable formulation when administered at about a 100 mg dose, but the time to reach maximum concentration of and eliminate trimethobenzamide are similar for the oral and intramuscular (I.M.) dosage forms. In addition, an oral pediatric dose of about 120 mg of trimethobenzamide of an oral formulation in accordance with the present invention is uniquely approximately bioequivalent to a FDA-approved 200 mg intramuscular (I.M.) trimethobenzamide HCl injectable formulation when administered at a dose of about 100 mg.

By the terms “bioequivalent”, “bioequivalence”, and “bioequivalency”, they are used herein interchangeably to describe pharmaceutical equivalent products that display comparable bioavailability when studied under similar experimental conditions. These terms are also used herein consistent with the definitions and concepts assigned to them under the U.S. Drug Price Competition and Patent Term Restoration Act of 1984, including the conditions set forth in § 550(j)(7)(B), and 21 CFR § 320.24, which are incorporated herein by reference in their entirety.

Thus, bioequivalence, as used herein, refers to the equivalent release of the same drug substance from two or more drug products or formulations which leads to an equivalent rate and extent of absorption from these products or formulations. In other words, if a drug product contains a drug substance that is chemically identical and is delivered to the site of action at the same rate and extent as another drug product, then it is equivalent. Methods to define bioequivalence can be found in 21 CFR 320.24, and include (1) pharmacokinetic (PK) studies, (2) pharmacodynamic (PD) studies, (3) comparative clinical trials, and (4) in-vitro studies, which are incorproated herein by reference in their entireties. Of course, the choice of study used, such as illustrated herein in Example 1, is based upon the site of action of the drug and the ability of the study design to compare drug delivered to that site by the two products.

By the term, “bioavailability”, it too refers to the definition and concepts assigned to this term under the Drug Price Competition and Patent Term Restoration Act of 1984, in particular in §550(j)(8)(B) and is used herein consistent with such definition and concept, which is incorporated herein by reference in its entirety.

The oral compositions of the present invention are useful for providing relief to patients experiencing a nausea and/or an emetogenic condition. The oral compositions of the present invention are also effective for providing palliative management of nausea and vomiting. The oral trimethobenzamide compositions are efficacious in patients undergoing, about to undergo, or recovering from chemotherapy for a deadly disease, such as cancer. However, other blood-borne and afferent conditions, such as, vertigo, motion sickness, AIDS, food poisoning, radiation, and other acute or chronic diseases and infections that cause nausea, emesis, or associated symptoms thereof, may be effectively treated and managed by the administration of the oral trimethobenzamide compositions disclosed herein. In particular, the oral trimethobenzamide compositions of the invention find exceptional beneficial use in patients experiencing nausea induced by gastroenteritis or nausea and vomiting in postoperative patients. In these patients (no matter what the cause of their illness) the composition provides relief of unwanted symptoms of nausea, vomiting and the like. Thus, the oral trimethobenzamide compositions of the present invention are effective for controlling nausea and vomiting in adults and children.

By the terms “control” or “controlling”, when used in connection with nausea and vomiting herein, they are used interchangeably and refer to a reduction in the incidence or severity of symptoms associated with nausea and vomiting.

Dosage forms of the present invention can be manufactured by standard manufacturing techniques. For example, in one manufacture the trimethobenzamide and the pharmaceutical diluents are blended and pressed into a solid layer. In another example of manufacture, the dosage form is manufactured by the wet granulation technique. In the wet granulation technique, for example, the trimethobenzamide and water are blended together to form a granulation. Other acceptable granulating fluids, such as various alcohols like methanol and ethanol, or other suitable organic solvents, may also be used for this purpose. The wet granulation is then dried overnight. The dried granulate is then blended with the other pharmaceutical ingredients to form a pre-blended master blend. The pre-blend master blend is then passed through a suitable mesh screen to generate a master blend. Capsules may then be filled from the master blend to form the desired oral dosage of trimethobenzamide. Alternatively, the master blend may be used to form other suitable oral dosage forms, e.g., tablets, caplets, liquid and powders.

The following examples are given by way of illustration only and are not to be considered limitations of this invention or many apparent variations of which are possible without departing from the spirit or scope thereof.

It should be understood that in Examples 1-4 that follow, the 300 mg trimethobenzamide capsules referenced therein were prepared in accordance with Example 5 and have the formula set forth in Table 2 above.

### EXAMPLE 1

### Clinical Report for a Randomized, Single-Dose, Open-Labeled, Four-Way Crossover Study to Assess the Bioequivalence of Three Oral Tigan® (Trimethobenzamide Hydrochloride) Formulations Compared to Tigan® I.M. Injectable (Trimethobenzamide Hydrochloride) Formulation

The primary objective of this study was to assess the bioavailability and to determine the bioequivalence of three oral trimethobenzamide hydrochloride formulations compared to the 200 mg intramuscular (I.M.) injectable formulation in order to determine the oral dose of trimethobenzamide hydrochloride that would be plasma (exposure) equivalent to the 200 mg intramuscular injectable formulation.

This study was a randomized, single-dose, open-labeled, four-way crossover bioavailability and bioequivalence study. A minimum five day washout was required between treatment periods.

Table 3 summarizes the disposition of study subjects. Seventy-four healthy, non-smoking, male & female subjects, 18-65 years old were enrolled in this study. A total of 68 subjects completed all the requirements of the protocol. There were no enrollment violations. However, nine of the 74 subjects began the study during the third period of the initial group of subjects. All 74 subjects who received at least one dose of study medication were included in the safety analysis.

Subjects were advised that they were free to withdraw from the study at any time. The Investigator or Sponsor could also withdraw a subject from the study at any time, if the subject became ill or his/her behavior compromised the outcome of the study. Any withdrawal of subjects from the study was documented in the final study report.

This study was a single center, randomized, open-label, 4-way crossover study in healthy subjects. Dosing was administered in the fasted state. Seventy-four (74) subjects were randomized into the study. All seventy-four subjects received one or more doses of study medication and are therefore included in the safety assessment. On Treatment Day 1 of the first treatment period, each subject was randomized to receive one of four possible study treatment sequences. The treatment groups consisted of the four treatments administered sequentially as DCAB, ADBC, BACD or CBDA. These treatments are shown below.

| | |
|---|---|
| Treatment A: | Oral trimethobenzamide hydrochloride capsules, 400 mg |
| | (4 × 100 mg capsule) |
| Treatment B: | Oral trimethobenzamide hydrochloride capsules, 300 mg |
| | (1 × 300 mg capsule) |
| Treatment C: | Oral trimethobenzamide hydrochloride capsules, 400 mg |
| | (1 × 400 mg capsule) |
| Treatment D: | I.M. injectable trimethobenzamide hydrochloride, 200 mg |
| | (2 ml × 100 mg/ml) |

Each treatment period was separated by a minimum of a 5 day washout. Table 3 presents a summary of the extent of drug exposure.

TABLE 3
Disposition of Subjects

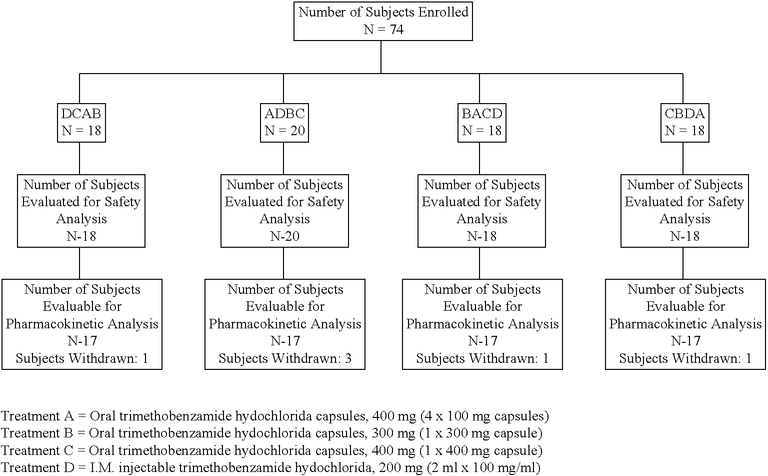

There were no significant differences between the demographic data between the study subjects assessed for safety compared to those who were eligible for the pharmacokinetic analysis.

A signed informed consent was obtained from each subject. For screening purposes, the following procedures were required of each subject and were performed by the clinical site within 30 days prior to the study: a complete physical examination including a medical history, vital signs, clinical laboratory safety tests on blood and urine, medications taken prior to and during the study, and a serum pregnancy test for female subjects.

During the treatments, temperature, blood pressure, heart rate and respiratory rate were monitored as a safety measure. A physical examination and clinical laboratory tests were repeated at the completion of the study. Subjects were observed and questioned throughout the study for the occurrence of any adverse events.

One subject took throat lozenges prior to check-in. Another subject did not have a serum pregnancy test performed at screening; however, she had been postmenopausal for 3 years. These protocol exceptions were granted. During the study, two subjects did not remain seated for 4 hours after dosing as required; however, there was no impact on the study. Five (5) subjects failed to return for repeat post study labs. Attempts to contact them were documented appropriately; they were considered lost to follow-up.

A blood sample was collected from each subject by venipuncture using a 7-ml K₃-EDTA collection tube prior to each treatment (0 hour). Additional blood samples were taken at about 0.083, 0.25, 0.5, 0.75, 1, 1.25, 1.5, 2, 2.5, 3, 4, 6, 8, 12, 16 and 24 hours post-dose. Plasma harvested from these blood samples was used for the assay of trimethobenzamide hydrochloride plasma concentrations.

Sample collections were considered deviations if they were not drawn within about 5 percent of the scheduled time. There were a total of 154 sampling time deviations. Seventy-six (76) pre-dose samples were collected more than 0.5 hour prior to dosing with delayed drug administration cited as the primary reason. Of the 78 late post dose draws, the majority of them occurred within the first 0.05 hours. The primary reasons for the delayed post-dose samples included difficult venous access and late draws. The actual sampling times were used in the PK analysis.

The plasma concentration-time data was used to determine the following pharmacokinetic parameters for each treatment: Cₘₐₓ (maximum trimethobenzamide hydrochloride plasma concentration), Tₘₐₓ. (time to Cₘₐₓ), AUCₗₐₛₜ (area under the plasma concentration-time curve up to the last quantifiable concentration), AUC_{0-inf}, (area under the plasma concentration-time curve extrapolated to infinity), and Kₑₗ (elimination phase rate constant). An analysis of variance (ANOVA) was performed on the log-transformed parameters AUCₗₐₛₜ, AUC_{0-inf}, and Cₘₐₓ, with treatment, period, sequence, and subject nested within sequence effects being evaluated.

Trimethobenzamide plasma concentrations were comparable for the 200 mg I.M. injection and the 300 mg capsule and for the 4×100 mg capsules and the 400 mg capsule. The time to reach maximum concentration was similar for all dosage forms. The elimination of trimethobenzamide, on average, was similar for all dosage forms.

The pharmacokinetic parameters for trimethobenzamide following the administration of a 200 mg I.M. injection, 300 mg capsule, 400 mg capsule and 4×100 mg capsules are presented in Table 4. Statistical comparisons of Cₘₐₓ, AUCₗₐₛₜ, and AUC_{0-inf} are presented in Table 5.

TABLE 4
Trimethobenzamide Noncompartmental Pharmacokinetic Parameters
Following the Administration of 200 mg I.M. Injection, 300 mg Oral
Capsule, 400 mg Oral Capsule and 4 × 100 mg Oral Capsules

| | | 200 mg I.M. | 300 mg Oral | 400 mg Oral | 4 × 100 mg Oral |
|---|---|---|---|---|---|
| Parameter | Statistic | Injection | Capsule | Capsule | Capsules |
| Cₘₐₓ (mcg/L) | Mean | 3728.79 | 3816.94 | 5211.23 | 5197.73 |
| | SD | 997.385 | 1355.016 | 1788.106 | 1534.570 |
| | Min | 2081.99 | 1665.96 | 1761.94 | 2379.82 |
| | Median | 3602.76 | 3679.59 | 4952.52 | 5150.37 |
| | Max | 6195.54 | 8961.81 | 9583.09 | 9807.35 |
| | CV% | 26.7 | 35.5 | 34.3 | 29.5 |
| AUCₗₐₛₜ (mcg*hr/L) | Mean | 10123.78 | 9460.65 | 12667.77 | 12426.04 |
| | SD | 1708.292 | 2429.683 | 3433.118 | 3335.331 |
| | Min | 7239.39 | 5831.78 | 6632.49 | 7176.55 |
| | Median | 9933.61 | 9136.86 | 12461.18 | 11574.82 |
| | Max | 14551.62 | 15966.59 | 23338.66 | 22586.65 |
| | CV% | 16.9 | 25.7 | 27.1 | 26.8 |
| AUC_{o-inf} (mcg*hr/L) | Mean | 10462.00 | 10218.11 | 13647.39 | 13493.38 |
| | SD | 1807.731 | 2690.333 | 3760.144 | 3694.251 |
| | Min | 7425.20 | 6186.70 | 6934.15 | 7632.61 |
| | Median | 10169.03 | 9935.72 | 13241.34 | 12541.88 |
| | Max | 16007.42 | 17661.15 | 25564.76 | 24700.70 |
| | CV% | 17.3 | 26.3 | 27.6 | 27.4 |
| Kₑₗ (hr⁻¹) | Mean | 0.11 | 0.10 | 0.10 | 0.09 |
| | SD | 0.025 | 0.027 | 0.025 | 0.022 |
| | Min | 0.06 | 0.05 | 0.05 | 0.05 |
| | Median | 0.11 | 0.10 | 0.10 | 0.10 |
| | Max | 0.17 | 0.17 | 0.16 | 0.14 |
| | CV% | 23.6 | 27.9 | 24.8 | 23.6 |
| T_{1/2} (hr) | Mean | 6.83 | 7.82 | 7.42 | 8.02 |
| | SD | 1.738 | 2.371 | 2.020 | 2.318 |
| | Min | 4.13 | 4.13 | 4.29 | 4.96 |
| | Median | 6.45 | 7.16 | 7.10 | 7.28 |
| | Max | 11.46 | 15.17 | 13.81 | 14.79 |
| | CV% | 25.5 | 30.3 | 27.2 | 28.9 |
| Tₘₐₓ (hr) | Mean | 0.54 | 0.78 | 0.73 | 0.66 |
| | SD | 0.214 | 0.244 | 0.223 | 0.200 |
| | Min | 0.25 | 0.50 | 0.50 | 0.25 |
| | Median | 0.50 | 0.75 | 0.75 | 0.73 |
| | Max | 1.00 | 1.50 | 1.50 | 1.25 |
| | CV% | 40.1 | 31.3 | 30.4 | 30.2 |
| F_{AUClast}* | Mean | N/A | 0.62 | 0.62 | 0.61 |
| | SD | N/A | 0.090 | 0.111 | 0.099 |
| | Min | N/A | 0.34 | 0.29 | 0.38 |
| | Median | N/A | 0.63 | 0.62 | 0.60 |
| | Max | N/A | 0.78 | 0.84 | 0.86 |
| | CV% | N/A | 14.6 | 17.8 | 16.2 |
| F_{AUC0-inf}* | Mean | N/A | 0.65 | 0.65 | 0.64 |
| | SD | N/A | 0.093 | 0.116 | 0.107 |
| | Min | N/A | 0.35 | 0.30 | 0.38 |
| | Median | N/A | 0.66 | 0.65 | 0.62 |
| | Max | N/A | 0.81 | 0.85 | 0.88 |
| | CV% | N/A | 14.3 | 17.9 | 16.7 |

* = F relative to 200 mg I.M. Injection
N/A = not applicable

TABLE 5
Mean Ratios and 90% Confidence Intervals for Trimethobenzamide
from Treatment Comparisons Based on Log Transformed Cₘₐₓ,
AUCₗₐₛₜ, and AUC_{inf}

| Comparison | | | | |
|---|---|---|---|---|
| (Test vs. | | Cₘₐₓ | AUCₗₐₛₜ | AUC_{0-inf} |
| Reference) | Statistic | (mcg/L) | (mcg*hr/L) | (mcg*hr/L) |
| A vs. D | Mean Ratio | 138.0 | 120.1 | 126.0 |
| | 90% | 129.5-146.9 | 116.8-123.4 | 122.5-129.6 |
| | Confidence | | | |
| | Interval | | | |
| B vs. D | Mean Ratio | 100.0 | 91.9 | 96.0 |
| | 90% | 93.9-106.5 | 89.5-94.5 | 93.3-98.7 |
| | Confidence | | | |
| | Interval | | | |
| C vs. D | Mean Ratio | 136.0 | 122.2 | 127.5 |
| | 90% | 127.7-144.9 | 118.9-125.6 | 124.0-131.2 |
| | Confidence | | | |
| | Interval | | | |

A = 4 × 100 mg Oral Capsules
B = 300 mg Oral Capsule
C = 400 mg Oral Capsule
D = 200 mg I.M. Injection

The mean maximum concentration, Cₘₐₓ, was comparable following the administration of the 200 mg I.M. injection and the 300 mg capsule, with mean±SD maximum plasma trimethobenzamide concentrations of 3728.79±997.385 mcg/L and 3816.94±1355.016 mcg/L for the 200 mg I.M. injection and 300 mg capsule, respectively. Following the administration of the 4×100 mg capsules and the 400 mg capsule, the mean Cₘₐₓ was approximately 39% greater than that following the 200 mg I.M. injection, with mean±SD maximum plasma trimethobenzamide concentrations of 5197.73±1534.570 mcg/L and 5211.23±1788.106 mcg/L, for the 4×100 mg capsules and the 400 mg capsule, respectively. The geometric least squares means ratios and 90% confidence intervals for Cₘₐₓ for each oral dosage form using the 200 mg I.M. injection as the test product were 100.0 (93.9%-106.5%), 136.0 (127.7%-144.9%), and 138.0 (129.5%-146.9%) for the 300 mg capsule, 400 mg capsule, and the 4×100 mg capsules, respectively. The Tₘₐₓ was similar for all dosage forms with a median (range) of 0.50 (0.25-1.00) hr, 0.75 (0.50-1.50) hr, 0.75 (0.50-1.50) hr, and 0.73 (0.25-1.25) hr, for the 200 mg I.M. injection, 300 mg capsule, 400 mg capsule, and the 4×100 mg capsules, respectively.

The measures of exposure, AUCₗₐₛₜ and AUC_{0-inf}, were comparable for the 200 mg I.M. injection and the; 300 mg capsule as evidenced by mean±SD values for AUCₗₐₛₜ of 10123.78±1708.292 mcg*hr/L and 9460.65±2429.683 mcg*hr/L and AUC_{0-inf} of 10465.00±1807.731 mcg*hr/L and 10218.11±2690.333 mcg*hr/L for the 200 mg I.M. injection and the 300 mg capsule, respectively. The mean AUCₗₐₛₜ and AUC_{0-inf} following the administration of the 4×100 mg capsules and the 400 mg capsule were approximately 20% greater than that following the 200 mg I.M. injection, with mean±SD values for AUCₗₐₛₜ of 12426.04±3335.331 mcg*hr/L and 12667.77±3433.118 mcg*hr/L and AUC_{0-inf} of 13493.38±3694.251 mcg*hr/L and 13647.39±3760.144 mcg*hr/L for the 4×100 mg capsules and the 400 mg capsule, respectively. The geometric least squares means ratios and 90% confidence intervals for AUCₗₐₛₜ and AUC_{0-inf} for each oral dosage form using the 200 mg I.M. injection as the test product were 91.9 (89.5%-94.5%), 122.2 (118.9%-125.6%), and 120.1 (116.8%-123.4%) for AUCₗₐₛₜ and 96.0 (93.3%-98.7%)., 127.5 (124.0%-131.2%), and 126.0 (122.5%-129.6%) for AUC_{0-inf} for the 300 mg capsule, 400 mg capsule, and the 4×100 mg capsules, respectively. The results of the ANOVA indicated that there was not a significant sequence effect indicating that the order of treatment had no effect on the outcome of the study.

The mean±SD bioavailability (F) of the oral dosage forms relative to the 200 mg I.M. injection using AUCₗₐₛₜ and AUC_{0-inf} were 0.62±0.090, 0.62±0.111, and 0.61±0.099 for AUCₗₐₛₜ and 0.65±0.093, 0.65±0.116, and 0.64±0.107 for AUC_{0-inf} for the 300 mg capsule, 400 mg capsule, and the 4×100 mg capsules, respectively.

The elimination half-life, T_{1/2}, was similar across all dosage forms with mean±SD values for the 200 mg I.M., 300 mg capsule, 400 mg capsule, and 4×100 mg capsules of 6.8±1.74 hr, 7.8±2.37 hr, 7.4±2.02 hr, and 8.0±2.32 hr, respectively.

A total of 28 adverse events (AEs) were experienced; 27 were treatment emergent. Twenty-one (21) of these were assessed as related to study treatment. All related AEs are known to occur with trimethobenzamide and were primarily mild or moderate in intensity. One (1) severe AE occurred in one subject which was judged as unrelated to study treatment-severe pharyngitis. There were no serious AEs. The type or severity of AEs reported during the four treatment periods did not appear to differ significantly. A summary of treatment emergent adverse events (AEs) is presented in Table 6 using the preferred medical term.

TABLE 6
Summary of Treatment Emergent Adverse Events by Randomization Sequence

| | A D B C | | B A C D | | C B D A | | D C A B | |
|---|---|---|---|---|---|---|---|---|
| | (N = 20) | | (N = 18) | | (N = 18) | | (N = 18) | |
| | n | % | n | % | n | % | n | % |
| Number of Subject With At Least | 5 | 25.0 | 4 | 22.2 | 4 | 22.2 | 5 | 27.8 |
| one AE | | | | | | | | |
| Number of Subject With No AE | 5 | 75.0 | 14 | 77.8 | 14 | 77.8 | 13 | 72.2 |
| HEADACHE | 1 | 5.0 | 0 | 0.0 | 3 | 16.7 | 3 | 16.7 |
| DIZZINESS | 3 | 15.0 | 0 | 0.0 | 0 | 0.0 | 0 | 0.0 |
| PARESTHESIA | 0 | 0.0 | 2 | 11.1 | 0 | 0.0 | 1 | 5.6 |
| ABDOMINAL PAIN | 1 | 5.0 | 0 | 0.0 | 0 | 0.0 | 0 | 0.0 |
| CONJUNCTIVITIS | 1 | 5.0 | 0 | 0.0 | 0 | 0.0 | 0 | 0.0 |
| DIARRHEA | 0 | 0.0 | 0 | 0.0 | 1 | 5.6 | 0 | 0.0 |
| EDEMA, PALMAR | 1 | 5.0 | 0 | 0.0 | 0 | 0.0 | 0 | 0.0 |
| ERYTHEMA, EXTERNAL NOSE | 0 | 0.0 | 0 | 0.0 | 1 | 5.6 | 0 | 0.0 |
| ERYTHEMA, PALMAR | 1 | 5.0 | 0 | 0.0 | 0 | 0.0 | 0 | 0.0 |
| HYPOTONIA | 0 | 0.0 | 0 | 0.0 | 1 | 5.6 | 0 | 0.0 |
| INJECTION SITE REACTION | 0 | 0.0 | 1 | 5.6 | 0 | 0.0 | 0 | 0.0 |
| LARYNGISMUS | 0 | 0.0 | 1 | 5.6 | 0 | 0.0 | 0 | 0.0 |
| LEG CRAMPS | 0 | 0.0 | 0 | 0.0 | 0 | 0.0 | 1 | 5.6 |
| NAUSEA | 1 | 5.0 | 0 | 0.0 | 0 | 0.0 | 0 | 0.0 |
| NAUSEA AND VOMITING | 1 | 5.0 | 0 | 0.0 | 0 | 0.0 | 0 | 0.0 |
| PHARYNGITIS | 0 | 0.0 | 0 | 0.0 | 0 | 0.0 | 0 | 0.0 |
| RASH | 0 | 0.0 | 1 | 5.6 | 0 | 0.0 | 0 | 0.0 |

*Only the first occurrence of each adverse event is reported for each subject in each treatment group
A = oral trimethobenzamide hydrochloride capsules, 400 mg (4 × 100 mg capsules)
B = oral trimethobenzamide hydrochloride capsules, 300 mg (1 × 300 mg capsule)
C = oral trimethobenzamide hydrochloride capsules, 400 mg (1 × 400 mg capsule)
D = I.M. trimethobenzamide hydrochloride, 200 mg (2 ml × 100 mg/ml)

Neurologic complaints were the most frequent (15), with 8 headaches seen in a total of 7 subjects, 2 of which were assessed as unrelated to study treatment. The other 6 headaches had a possible relationship and were primarily of mild intensity. The next most frequent neurologic complaints were dizziness (3) and paresthesias (3). The incidence of neurologic-related AEs is predictable given that this is a known adverse event with trimethobenzamide hydrochloride. The incidence of AEs was not significantly different for other body systems. There were no apparent differences in the incidence of AEs noted among the 4 treatment periods.

The most severe occurrence of an AE is reported for subjects reporting the same AE more than once.

The incidence of AEs by relationship to study drug is similar between the treatment groups. As described previously, the most frequently reported AE in all 4 treatment groups was headache of primarily mild intensity: 6 were classified as being possibly related and 2 as being unrelated. Two (2) of the headaches judged possibly related were experienced by the same subject in 2 separate treatment periods and were of moderate and mild intensity, respectively. The 3 episodes of dizziness were classified as moderate: 2 had a possible relationship and 1 had a probable relationship. Three (3) episodes of paresthesias were experienced; all were of mild intensity and possibly related to study treatment.

The majority of the remaining AEs were reported as primarily mild (10) with significantly fewer events classified as moderate (4). Of the 10 mild AEs: 2 were unrelated; 5 were possibly related and 3 were probably related. Of the 4 moderate AEs: 1 was unrelated; 1 was possibly related and 2 were probably related. Only one AE in the study was classified as “severe”. One subject, randomized to treatment sequence DCAB, experienced pharyngitis which was classified as severe, which was determined to be unrelated to study treatment and which resolved the following day without any sequelae.

Six (6) subjects were prematurely withdrawn from the study. Four (4) of these 6 were due to adverse events. One (1) subject was withdrawn due to noncompliance after the second dosing period. The sixth subject was dismissed after the first dosing period due to difficult blood draws.

Of the four subjects that prematurely withdrew, one subject received Treatment B (oral trimethobenzamide hydrochloride capsules, 300 mg) in treatment period 3 of the study on Jun. 10, 2000. She developed mild conjunctivitis on Jun. 14, 2000, which was treated with sodium sulfacetamide and resolved on Jun. 24, 2000 without sequelae. Though this event was determined as unrelated to study treatment, the subject chose not to continue participation in the study.

The second of the four subjects that prematurely withdrew received Treatment B (oral trimethobenzamide hydrochloride capsules, 300 mg) in treatment period 1 of the study on May 27, 2000 without incident. She developed a rash on May 31, 2000 prior to Period 2 dosing. She received Treatment A (oral trimethobenzamide hydrochloride capsules, 400 mg) in treatment period 2 of the study on Jun. 3, 2000. Subsequent to Period 2 dosing, the rash was classified as moderate and treated with Benadryl. The rash resolved on Jun. 18, 2000 without sequelae. No concomitant medications, in addition to Benadryl, were reported as having been taken. This event was determined to have a probable relationship to study treatment and the subject was dismissed from the study prior to Period 3 dosing.

The third of the four subjects that prematurely withdrew received Treatment A (oral trimethobenzamide hydrochloride capsules, 400 mg) in treatment period 1 of the study at 11:37 hours on May 27, 2000. She experienced moderate nausea and vomiting at 16:00 hours on May 27, 2000, which resolved on Jun. 4, 2000 without sequelae. No medical intervention was necessary. This AE was determined to have a probable relationship to study treatment. The subject was discontinued from further participation in the study.

The fourth of the four subjects that prematurely withdrew received Treatment A (oral trimethobenzamide hydrochloride capsules, 400 mg) in treatment period 3 of the study on Jun. 10, 2000 without incident. She developed a leg cramp of mild intensity at 02:45 hours on Jun. 17, 2000 approximately 5 hours prior to dosing for treatment period 4. She received Treatment B (oral trimethobenzamide hydrochloride capsules, 300 mg) in treatment period 4 of the study at 08:37 hours on Jun. 17, 2000. Approximately 1 hour after dosing, she chose to discontinue further participation in the study. The cramp resolved without sequelae approximately 16 hours after onset at 19:00 hours on Jun. 17, 2000. No concomitant medications were reported as having been taken. No medical intervention was necessary. This AE was determined to have a possible relationship to study treatment.

All abnormal laboratory findings were determined to be clinically acceptable. Hyperkalemia was noted in 55 of the 72 samples drawn at the end of the study. All 55 hyperkalemic results were due to hemolyzed specimens and determined to be not clinically significant. Only 2 subjects returned for repeat testing; serum potassium levels were within normal limits. All potassium levels measured at baseline were within normal limits.

Thirteen (13) percent of subjects who had a normal hemoglobin level prior to treatment had low hemoglobin at the end of treatment with a mean change of 0.6 g/dl. Eight (8) percent of subjects had a low hematocrit prior to treatment compared to 34 percent at the end of treatment with a mean change of 2.3 percent. A decreased red blood cell count was seen in 5 percent of subjects prior to treatment compared to 11 percent at the end of treatment; the mean change was 0.2×10¹²/L. These laboratory findings were determined to have no clinical significance.

No medical intervention was necessary for any abnormal laboratory finding.

The vital sign and physical examination findings were either within normal range or considered clinically acceptable.

Overall, trimethobenzamide hydrochloride appeared to be safe and well tolerated with no significant differences among the 4 treatment periods. There was also no difference between all study subjects (74) and those subjects (68) who were included in the pharmacokinetic analysis. Twenty-one (21) adverse events were determined to be related to study treatment; all were of mild to moderate intensity with neurologic complaints being the most frequent. Neurologic-related adverse events are well-known side effects of trimethobenzamide hydrochloride. There were no unexpected adverse events. No apparent significant differences in adverse events were noted among the 4 treatment periods.

As indicated above, this study was performed to assess the bioavailability and to determine the bioequivalence of three oral trimethobenzamide hydrochloride formulations compared to the intramuscular (I.M.) injectable formulation in healthy subjects in order to determine the oral dose of trimethobenzamide hydrochloride that would be plasma (exposure) equivalent to the injectable formulation.

The Cₘₐₓ, AUCₗₐₛₜ, and AUC_{0-inf}, for the 200 mg I.M. injection and 300 mg oral capsule were comparable as evidenced by geometric least squares means ratios and 90% confidence intervals that fell within the 80%-125% confidence limit for bioequivalence. In addition, the Tₘₐₓ was similar between all of the oral dosage forms as evidenced by median values that differed from the 200 mg I.M. injection by 15 minutes and ranges that overlapped. These results indicate a similar exposure between the 200 mg I.M. injection and the 300 mg capsule.

The mean Cₘₐₓ, AUCₗₐₛₜ, and AUC_{0-inf}, for the 400 mg capsule and the 4×100 mg capsules were approximately 20% larger than those values achieved following the 200 mg I.M. injection. The geometric least squares means ratios and 90% confidence intervals for the 400 mg capsule and the 4×100 mg capsules, using the 200 mg I.M. injection as the reference, did not fall within the 80%-125% confidence limit for bioequivalence. For both dosage forms, the geometric least squares means ratios were 120 and above for all three pharmacokinetic parameters. However, the Tₘₐₓ was similar between the dosage forms as evidenced by median values that differed from the 200 mg I.M. injection by 15 minutes and ranges that overlapped. These results indicated that the 400 mg capsules and the 4×100 mg capsules resulted in exposures that were greater than those achieved by the 200 mg I.M. injection.

The results from this study indicate that the 300 mg capsule results in a pharmacokinetic profile that is similar in terms of Cₘₐₓ, Tₘₐₓ, AUCₗₐₛₜ, and AUC_{0-inf}, to the 200 mg I.M. injection and demonstrates the equivalence of Cₘₐₓ, AUCₗₐₛₜ and AUC_{0-inf} between the 300 mg capsule and the 200 mg I.M. injection. In addition, all four dosage forms were safe and well tolerated. Based on these results, the 300 mg capsule is believed to be plasma (exposure) equivalent and yield an efficacy and safety profile similar to the 200 mg I.M. injection.

### EXAMPLE 2

### Protocol for Dissolution Profile of Trimethobenzamide Capsule 300 mg

This protocol directs the execution of a dissolution profile for Tigan 300 mg capsule. The profile is performed on 12 individual capsule units with data obtained at 15, 30, 45 and 60-minute timepoints. Each capsule data set is profiled individually, as illustrated in FIG. 1. The product lot number used for the profile is C002. The capsule formulas are set forth in Table 2.

Place about 900 ml of water into each vessel. Equilibrate the medium to about 37±0.5° C. Place one capsule in each apparatus 1 (Baskets), and immediately operate the apparatus at about 100 rpm. Pull a uniform aliquot from each of the vessels at about 15, 30, 45 and 60-minute timepoints. Filter each of the aliquots.

Weigh accurately approximately 50 mg of USP Trimethobenzamide HCl reference standard or equivalent and transfer with the aid of water into a 100 ml volumetric flask. Fill to volume with water and mix. Take about a 2.0 ml aliquot of the solution and transfer to a 100 ml volumetric flask. Fill to volume with water and mix.

Take about 3.0 ml of a filtered portion of the dissolution sample and transfer to a 100 ml volumetric flask. Fill to volume with water and mix.

Determine the amount of Trimethobenzamide HCl dissolved from ultraviolet absorbances at the wavelength of maximum absorbance at about 258 nm of the Sample Preparation in comparison with the Standard Preparation.

To calculate:

Au/As×C×100 mL/3 mL×900 mL/300 mg×100=% Trimethobenzamide HCl

Where:

Au=Absorbance of Sample

As=Absorbance of Standard

C=Concentration of Standard (mg/mL)

Note: Calculation is adjusted to account for each aliquot removed after the initial timepoint.

There are no acceptance criteria for the individual time points. The method specification reads NLT 75% (Q) is dissolved in 45 minutes.

Each of the 12 individual capsule data sets compare favorably with one another. All capsules had potency determinations of greater than 75% (Q) in 45 minutes. In other words, all 12 capsules demonstrated comparable dissolution profiles, i.e., the trimethobenzamide concentration strength (300 mg) in all 12 capsules dissolved by more than about 75% (Q) within about 45 minutes. More particularly, approximately 70% or more dissolved in about 15 minutes, approximately 95% or more dissolved in about 30 minutes, approximately 97% or more dissolved in about 45 minutes, and approximately all was dissolved in about 60 minutes. See FIG. 1 for data and profiles.

### EXAMPLE 3

### Assay for Trimethobenzamide Hydrochloride 300 mg Capsules

Take about 3.0 ml of a filtered portion of the dissolution sample and transfer to a 100 ml volumetric flask. Fill to volume with water and mix.

Determine the amount of C₂₁H₂₈N₂O₅.HCl dissolved from ultraviolet absorbances at the wavelength of maximum absorbance at about 258 nm of filtered portions of the solution under test, suitability diluted with Dissolution Medium, if necessary, in comparison with a Standard solution having a known concentration of USP Trimethobenzamide Hydrochloride RS in the same medium.

To calculate:

$$\frac{{A}_{u}}{{A}_{s}} \times C \times \frac{100 ml}{3 ml} \times \frac{900 ml}{300 mg \text{/} capsule} \times 100 = \% TMB \text{-} HCl$$

Where:

Aᵤ=Absorbance of sample

Aₛ=Absorbance of standard

C=Concentration of standard (mg/mL)

Weigh accurately 10 capsules individually, taking care to preserve the identity of each capsule. Remove the contents of each capsule by a suitable means. Weigh accurately the, emptied shells individually, and calculate for each capsule the net weight of its contents by subtracting the weight of the shell from the respective gross weight. From the results of the Assay, obtained as directed in this document, calculate the content of active ingredient in each of the capsules, assuming homogeneous distribution of the active ingredient.

Divide the average weight of capsule contents by the target weight and multiply by 100.

To prepare Capsule Sample, transfer, as completely as possible, the contents of not less than 20 trimethobenzamide 300 mg capsules to a suitable tared container, and determine the average weight per capsule. Mix the combined contents, and transfer an accurately weighed portion of the powder, equivalent to about 50 mg of trimethobenzamide hydrochloride, to a 100 ml volumetric flask. Add about 50 ml of dilute hydrochloric acid (1 in 120), shake the mixture for several minutes, then add dilute hydrochloric acid (1 in 120) to volume, and mix. Filter through small retentive filter paper (Whatman #3 or equivalent) or 0.45 μm nylon syringe filter, discarding the first 20 ml of the filtrate. Transfer about 4.0 ml of the subsequent filtrate to a 100 ml volumetric flask, add dilute hydrochloric acid (1 in 120) to volume, and mix well.

Concomitantly determine the absorbance of the Capsule Sample and the Standard Preparation using dilute hydrochloric acid (1 in 120) as the blank.

To calculate:

$$\begin{matrix}\frac{{A}_{u}}{{A}_{s}} \times C \times \frac{100 ml}{4 ml} \times 100 ml \times \frac{Avg . Wt .}{spl . Wt .} = mg TMB \text{-} HCl \\ \frac{mg TMB \text{-} HCl}{300 mg} \times 100 = \% Trimethobenzamide HCl \text{/} capsule\end{matrix}$$

Where:

Aᵤ=Absorbance of sample

Aₛ=Absorbance of standard

C =Concentration of standard (mg/ml)

According to this Example 3, the trimethobenzamide hydrochloride 300 mg capsules of the present invention have a potency of between about 90% and about 110%, of the theoretical 300 mg.

### EXAMPLE 4

### Assay—Trimethobenzamide HCl and Degradants

A suitable HPLC system consisting of a high pressure pump, injector, variable wavelength UV detector, and a data handling device is used.

Chromatographic Conditions:

| | |
|---|---|
| Column | MetaChem Inertsil, 4.6 × 250 mm column or |
| | equivalent |
| Mobile Phase | Acetonitrile:Buffer (20:80) with 3 ml/1000 ml |
| | Triethylamine, final pH of 3.4 +/− 0.05 |
| Flow Rate | about 1.0 ml/minute |
| Wavelength | 258 nm |
| Injection Volume | 100 μl |
| Temperature | Ambient |
| Approximate Run Time | 60 minutes |
| Relative Retention Times | Trimethobenzamide HCl-1.0 |
| | TMB Oxidation Product-1.4 |
| | 3,4,5-Trimethoxybenzoic Acid-approx. 1.9 |
| Column Wash | After each run, wash the column sequentially |
| | with a flow-rate of at least 1.5 ml/min, using |
| | HPLC-grade solvents as follows |
| | At least 10 minutes with HPLC-grade water |
| | At least 30 minutes with 80/20 Acetonitrile/ |
| | Water |
| | At least 10 minutes with 20/80 Acetonitrile/ |
| | Water |

Stability in Solution:

Standard: 5 days ambient temperature

Sample: 12 days ambient temperature

Mobile Phase:

For about 1000 ml, combine about 200 ml HPLC-grade Acetonitrile; 800 ml Buffer (see below), and 3 ml Triethylamine. Mix well, adjust the pH to about 3.4+/−0.05 with about 85% phosphoric acid, filter, and degas.

Buffer (about 20 mM Sodium Phosphate, Monobasic): For about 1000 ml, combine about 2.76 g of Sodium Phosphate, Monobasic with about 1000 ml of HPLC-grade water and mix until dissolved.

Standard Preparation of Trimethobenzamide HCl (TMB) Stock:

Accurately weigh about 30 mg of Trimethobenzamide HCl Reference Standard and transfer to a 50 ml volumetric flask. Dilute to volume with HPLC-grade water and mix until completely dissolved.

3,4,5 Trimethoxybenzoic Acid (TMBA) Stock:

Accurately weigh about 30 mg of 3,4,5-Trimethoxybenzoic Acid Reference Standard and transfer to a 250 ml volumetric flask. Dilute to volume with HPLC-grade water, sonicate until dissolved (this may take about 30 minutes or more), and mix well. Pipet about 5 ml of this solution into a 200 ml volumetric flask, dilute to volume with HPLC-grade water and mix well. Working Standard:

Pipet about 10 ml of the Trimethobenzamide HCl Stock and about 10 ml of the 3,4,5-Trimethoxybenzoic Acid Stock into a 100 ml volumetric flask. Dilute to volume with HPLC-grade water and mix well.

Sample Preparation

Initial Sample Solution:

Transfer, as completely as possible, the contents of not less than 20 Trimethobenzamide HCl Capsules to a suitable tared container and determine the average capsule content weight. Mix the combined contents and transfer an accurately weighed portion of the powder, approximately equivalent to the label claim of Trimethobenzamide HCl, to a 200 ml volumetric flask. Add about 100 ml of HPLC-grade water and sonicate for approximately 15 minutes. Cool to ambient temperature, dilute to volume with HPLC-grade water, and stir well. Transfer a portion of the sample to a centrifuge tube and centrifuge for approximately 20 minutes at a sufficient speed to pellet the particulates in the sample. The supernatant will be used to prepare the Working Sample.

Working Sample:

Pipet about 4 ml of the Initial Sample Solution into a 100-ml volumetric flask, dilute to volume with HPLC-grade water, and mix well.

System Suitability:

Chromatograph five replicate injections of the Working Standard and measure the peak areas of Trimethobenzamide HCl and 3,4,5-Trimethoxybenzoic Acid. The relative standard deviation does not exceed about 2.0% for Trimethobenzamide HCl or about 5.0% for 3,4,5-Trimethoxybenzoic Acid.

The tailing factor (T) of the Trimethobenzamide HCl and 3,4,5-Trimethoxybenzoic Acid peaks are not more than about 2.0 calculated by the following formula:

$$T = \frac{{W}_{0.05}}{2 ( f )}$$

Where:

W=the width of the peak measured at 5% of the peak height 0.05

f=the distance, at 5% of the peak height, from the peak maximum to the leading edge of the peak

Procedure

Inject 100 ul aliquots of Working Standard and Working Samples into a properly equilibrated liquid chromatograph and record the peak area responses.

To Calculate:

$$\begin{matrix}mg TMB = \frac{{Ar}_{w}}{{Ar}_{s}} \times {C}_{s} \frac{Avg CCW}{SW} \times 200 ml \times \frac{100 ml}{4 ml} \\ TMB as a \% of Label = \frac{mg TMB}{300 mg \text{/} cap} \times 100 \\ TMBA as a \% of TMB = \frac{{Ar}_{w}}{{Ar}_{t}} \times {C}_{t} \times \frac{( Avg CCW ) ( 200 ml ) ( 100 ml )}{( SW ) ( 4 ml ) ( 300 mg \text{/} cap )} \times 100\end{matrix}$$

Note: If the calculated result is less than the TMBA Limit of Quantitation of 0.14%, record as LT 0.14%. TMB Oxidation Product as a % of

$$TMB = \frac{{Ar}_{o}}{{Ar}_{w}} \times 100$$

Where:

Ar_{w}=Peak area of the TMB in Working Sample

Arₐ=Peak area of the TUBA in Working Sample

Arₒ=Peak area of TMB Oxidation Product in Working Sample

Arₛ=Average peak area TMB Working Standard

Arₜ=Average peak area TMBA Working Standard

Cₛ=TMB Working Standard concentration in mg/ml,

Cₜ=TMBA Working Standard concentration in mg/ml

CCW=Capsule content weight in mg

SW=Sample weight in mg

This Example 4 is a stability indicating chromatography method. It demonstrates how to calculate the potency of trimethobenzamide and the degradation components of trimethobenzamide, i.e., 3,4,5 trimethobenzoic acid and trimethobenzamide oxidation products. According to this Example 4, the Trimethobenzamide HCl Capsules (300 mg) demonstrated a potency of between about 90% and 110%, consistent with Example 3, and that 3,4,5 trimethobenzoic acid is present in concentrations of less than about 0.5% of the labeled content of trimethobenzamide and that minimally trace amounts of trimethobenzamide oxidation products were present thereby evidencing purity and potency for at least about 24 months.

### EXAMPLE 5

### Method of Manufacture of Trimethobenzamide Hydrochloride Capsules

A standard production formula or master blend, batch size approximately 120 Kg, which can be used to produce 300 mg and other strength trimethobenzamide capsules, is reported in Table 7 below.

TABLE 7
Standard Production Formula (Master Blend)

| Ingredient | Quantity | Unit | Quantity Per Gram |
|---|---|---|---|
| Trimethobenzamide | 81,888 | g | 0.6824 g |
| Hydrochloiride USP | | | |
| Lactose NF | 11,676 | g | 0.0973 g |
| Starch NF | 24,816 | g | 0.2068 g |
| Magnesium Stearate NF | 01,620 | g | 0.0135 g |
| Purified Water USP | 07,370 | g | —* |
| Total Weight Assuming Dryness | 120,000 | g | 1.0000 g |

*Used to granulate, does not appear in final product

Transfer 88,188 g of trimethobenzamide hydrochloride HCl USP to a Pony Mixer-Single or Tub Fast, Blade Fast, or other suitable mixers and blend for approximately 5-10 minutes. Continue mixing and granulate the blended trimethobenzamide hydrochloride HCl USP with approximately 7,370 ml of purified water USP. The purified water should be added very slowly. Continue mixing until a suitable granulation is achieved. If a suitable granulation is not achieved, gradually add an appropriate amount of purified water USP very slowly until a suitable granulation is achieved.

Evenly spread granulation on polyethylene lined Lydon Dryer trays or other suitable tray dryers. Dry at about 115° F.±5° F. overnight in a dryer.

Screen the granulated trimethobenzamide hydrochloride USP, approximately 81,888 g, together with approximately 24,816 g of Starch NF, approximately 1,620 g of Magnesium Stearate NF, and about 11,676 g of Lactose NF through a Fitzmill #1-A (0040) perforated plate, high speed, knives to form a pre-blended master blend.

Weigh and record the pre-blended master blend. The pre-blended master blend at this point in the manufacturing process should weigh approximately 120,000 g.

Transfer the pre-blended master blend, approximately 120,000 g, to a Ribbon Blender or other suitable blenders and blend for about 20 minutes to form a Master Blend, i.e., the Standard Production Formula set forth in Table 7 above. Transfer the Master Blend, approximately 120,000 g, to tared polyethylene lined containers.

To make oral capsules in accordance with the present invention, encapsulate the Standard Production Formula into capsules on an auger-fill capsule filling machine.

Using an auger-fill encapsulator equipped with selected size change parts which correspond to the size of the capsule selected to be filled, e.g., capsule size #1, perform set-ups by opening the capsules, filling the capsules with a predetermined amount of the Standard Production Formula of Table 7 as set forth in Table 8, and then closing the capsules to produce oral capsules with a desired trimethobenzamide strength.

TABLE 8
Trimethobenzamide Capsule Characteristics

| | Standard | | | |
|---|---|---|---|---|
| | Production | | | |
| Trimetho- | Formula | | | Master |
| benzamide | (Master | Batch Size | | Blend per |
| Capsule | Blend) | Number of | Capsule | Capsule |
| Strength | Table 7 | Capsules | Size & Color | Table 7 |
| 300 mg | 120,000 g | 270,270 | #1, Deep Purple #5 | 0.4440 g |
| 325 mg | 120,000 g | 249,480 | #1, #1EI or #0, | 0.4810 g |
| | | | Deep Purple #5 | |
| 350 mg | 120,000 g | 231,660 | #1, #1EI or #0, | 0.5180 g |
| | | | Deep Purple #5 | |
| 375 mg | 120,000 g | 216,216 | #1, #1EI or #0, | 0.5550 g |
| | | | Deep Purple #5 | |
| 400 mg | 120,000 g | 202,702 | #0, Deep Purple #5 | 0.5920 g |
| 425 mg | 120,000 g | 190,779 | #0, #0EI or #00, | 0.6290 g |
| | | | Deep Purple #5 | |
| 450 mg | 120,000 g | 180,180 | #0, #0EI or #00, | 0.6660 g |
| | | | Deep Purple #5 | |

To make 300 mg trimethobenzamide hydrochloride capsules, approximately 120,000 g of the Standard Production Formula (Master Blend) recited in Table 7 and 270,270 capsules, e.g., capsule size #1, capsule color Deep Purple #5, are used. Each #1 size, Deep Purple #5 capsule, when filled, will contain approximately 0.4440 g of the Standard Production Formula and approximately 300 mg of trimethobenzamide hydrochloride. If a different capsule strength is desired, e.g., 400 mg., again approximately 120,000 g of the Standard Production Formula (Master Blend) and 202,702 capsules, e.g., capsule size #0, capsule color Deep Purple #5, are used. In this case, each # 0 size, Deep Purple #5 capsule, when filled, will contain approximately 0.592 g of the Standard Production Formula and approximately 400 mg of trimethobenzamide hydrochloride.

To make pediatric trimethobenzamide hydrochloride capsules having a dosage strength of about 120 mg, again approximately 120,000 g of the Standard Production Formula (Master Blend) and 675,675 capsules, e.g., capsule size #4 are used. In this case, each #4 size capsule, when filled, will contain approximately 0.1776 g of the Standard Production Formula and approximately 120 mg of trimethobenzamide hydrochloride. See Table 9. If a different capsule strength is desired, e.g., 150 mg., again approximately 120,000 g of the Standard Production Formula (Master Blend) and 540,540 capsules, e.g., capsule size #3 or #4 el are used. In this case, each #3 or #4 el size capsule, when filled, will contain approximately 0.2220 g of the Standard Production Formula and approximately 150 mg of trimethobenzamide hydrochloride. See Table 9.

TABLE 9
Pediatric Trimethobenzamide Capsule Characteristics

| Pediatric | Standard | | | |
|---|---|---|---|---|
| Trimetho- | Production | | | |
| benzamide | Formula | Batch Size | | Master Blend |
| Capsule | (Master Blend) | Number of | | per Capsule |
| Strength | Table 7 | Capsules | Capsule Size | Table 7 |
| 120 mg | 120,000 g | 675,675 | #4 | 0.1776 g |
| 125 mg | 120,000 g | 648,648 | #3, #4 el, #4 | 0.1850 g |
| 130 mg | 120,000 g | 623,700 | #3, #4 el | 0.1924 g |
| 150 mg | 120,000 g | 540,540 | #3, #4 el | 0.2220 g |
| 175 mg | 120,000 g | 463,320 | #2, #3 el, #3 | 0.2590 g |
| 200 mg | 120,000 g | 405,405 | #2, #3 el | 0.2960 g |

Accordingly, it will be understood that embodiments of the present invention have been disclosed by way of example and that other modifications and alterations may occur to those skilled in the art without departing from the scope and spirit of the appended claims. Thus, the invention described herein extends to all such modifications and variations as will be apparent to the reader skilled in the art, and also extends to combinations and sub-combinations of the features of this description and the accompanying FIG. 1.

It will also be understood that, although preferred embodiments of the present invention have been illustrated in the accompanying FIG. 1 and described in the foregoing detailed description and examples, the invention is not limited to the embodiments disclosed, but is capable of numerous rearrangements, modifications and substitutions without departing from the spirit of the invention as set forth and defined by the following claims.

Having described our invention,

We claim:

1) An oral pediatric trimethobenzamide composition for treating and controlling nausea and/or vomiting in a child comprising
trimethobenzamide and a suitable pharmaceutical excipient, wherein said oral pediatric trimethobenzamide composition is at least about as effective as a 200 mg intramuscular (I.M.) trimethobenzamide HCl injectable formulation when administered in a dose of about 100 mg to treat and control nausea and/or vomiting.

2) An oral pediatric trimethobenzamide composition of claim 1, wherein said trimethobenzamide is present in an amount greater than 120 mg.

3) an oral pediatric trimethobenzamide composition of claim 2, wherein said trimethobenzamide is present in an amount selected from the group consisting of about 125 mg, 130 mg, 140 mg, 150 mg, 160 mg, 175, mg, 180 mg and 200 mg:

4) An oral pediatric trimethobenzamide composition of claim 1, wherein said trimethobenzamide is trimethobenzamide hydrochloride.

5) An oral pediatric trimethobenzamide composition of claim 1, wherein mean PK parameters for said oral pediatric trimethobenzamide composition are greater than mean PK parameters for the 200 mg intramuscular (I.M.) trimethobenzamide HCl injectable formulation formulation when administered in a dose of about 100 mg.

6) An oral pediatric trimethobenzamide composition of claim 1, wherein said oral pediatric trimethobenzamide composition is produced by the process of
granulating the trimethobenzamide, and
blending said pharmaceutical excipient with the granulated trimethobenzamide.

7) An oral pediatric trimethobenzamide composition of claim 1, wherein said oral pediatric trimethobenzamide composition is produced by the process of
mixing the trimethobenzamide with said pharmaceutical excipient.

8) An oral pediatric trimethobenzamide composition of claim 1, wherein said oral pediatric trimethobenzamide composition contains more than one pharmaceutical excipient.

9) An oral pediatric trimethobenzamide composition of claim 8, wherein said pediatric pharmaceutical excipients are starch, lactose and magnesium stearate.

10) An oral pediatric trimethobenzamide composition for treating and controlling nausea and/or vomiting in a child comprising
trimethobenzamide and a suitable pharmaceutical excipient, wherein said oral trimethobenzamide composition is approximately bioequivalent to a 200 mg intramuscular (I.M.) trimethobenzamide HCl injectable formulation when administered in a dose of about 100 mg to treat and control nausea and/or vomiting.

11) An oral pediatric trimethobenzamide composition of claim 10, wherein said trimethobenzamide is present in an amount of about 120 mg.

12) An oral pediatric trimethobenzamide composition of claim 10, wherein said trimethobenzamide is trimethobenzamide hydrochloride.

13) An oral pediatric trimethobenzamide composition of claim 10, wherein mean PK parameters for said composition are approximately equivalent to mean PK parameters for the 200 mg intramuscular (I.M.) trimethobenzamide HCl injectable formulation when administered in a dose of about 100 mg.

14) An oral pediatric trimethobenzamide composition of claim 10, wherein said oral pediatric trimethobenzamide composition is produced by the process of
granulating the trimethobenzamide, and
blending said pharmaceutical excipient with the granulated trimethobenzamide.

15) An oral pediatric trimethobenzamide composition of claim 10, wherein said oral pediatric trimethobenzamide composition is produced by the process of
mixing the trimethobenzamide with said pharmaceutical excipient.

16) An oral pediatric trimethobenzamide composition of claim 10, wherein said oral pediatric trimethobenzamide composition contains more than one pharmaceutical excipient.

17) An oral pediatric trimethobenzamide composition of claim 16, wherein said pharmaceutical excipients are starch, lactose and magnesium stearate.

18 ) An oral pediatric trimethobenzamide composition for treating and controlling nausea and/or vomiting in a child, said composition comprising
trimethobenzamide, and
a pharmaceutically acceptable excipient, wherein said oral trimethobenzamide composition, following oral administration to a warm-blooded animal, achieves plasma (exposure) levels which are at least approximately equal to or greater than those plasma (exposure) levels achieved by a FDA-approved 200 mg intramuscular (I.M.) trimethobenzamide HCl injectable formulation when administered intramuscularly to a warm-blooded animal at a dose of about 100 mg.

18) An oral pediatric trimethobenzamide composition of claim 1, wherein said trimethobenzamide is present in an amount of about 120 mg.

19) An oral pediatric trimethobenzamide composition of claim 10, wherein said trimethobenzamide is present in an amount greater than about 120 mg.
